(19) United States
(12) Patent Application Publication
(10) Pub. No.: US 2017/0007157 A1
(43) Pub. Date: Jan. 12, 2017

(54) ELECTRICAL-SIGNAL-BASED ELECTRODE-TISSUE CONTACT DETECTION

(71) Applicant: RAINBOW MEDICAL LTD., Herzliya (IL)

(72) Inventors: Yossi GROSS, Moshav Mazor (IL); Yehuda ZADOK, Kiryat Ono (IL); Assaf Israel Pele LEON, Petach Tikva (IL)

(21) Appl. No.: 14/794,737

(22) Filed: Jul. 8, 2015

(51) Int. Cl. A61B 5/06; A61B 5/0245; A61B 5/00; A61B 18/14; A61B 5/042

(52) U.S. Cl. CPC A61B 5/063; A61B 18/1492; A61B 5/0422; A61B 5/6885; A61B 5/7221; A61B 5/0245; A61B 2018/00434

(57) ABSTRACT

A method includes disposing a pair of current-application electrodes in or on a body, including disposing an intrarenal current-application electrode of the pair in a renal artery. Two or more sensing electrodes are disposed in or on the body, including disposing one of the sensing electrodes on en external surface of skin. Control circuitry is activated to (a) apply an electrical current between the pair of current-application electrodes, (b) while applying the electrical current, sense an electrical signal between the sensing electrodes, including the sensing electrode disposed on the external surface of the skin, and (c) based on the electrical signal, ascertain a level of contact between the intrarenal current-application electrode and a wall of the renal artery. In response to the level of contact being less than a threshold level of contact, a disposition of the intrarenal current-application electrode in the renal artery is adjusted.

### FIELD OF TEE APPLICATION

The present invention relates generally to electrode positioning techniques, and specifically to techniques for accurately positioning electrodes in the renal artery.

### BACKGROUND OF THE APPLICATION

Hypertension is a prevalent condition in the general population, particularly in older individuals. Sympathetic nervous pathways, such as those involving the renal nerve, are known to play a role in regulating blood pressure. Ablation of renal nerve tissue from the renal artery is a known technique for treating hypertension.

### SUMMARY OF THE APPLICATION

In some embodiments of the present invention, apparatus and methods are provided for ascertaining a level of contact between at least one intrarenal electrode disposed in a renal artery of a subject and a wall of the renal artery. If the level is not sufficient, a disposition the at least one intra renal electrode is typically adjusted.

In some applications of the present invention, control circuitry is configured to apply electrical pulses between a pair of electrodes, including at least one intrarenal electrode; calculate at least one time-varying component of electrode-tissue impedance based on applying the pulses; sense a periodic hemodynamic signal of the subject; calculate a level of correlation between the at least one time-varying component of the electrode-tissue impedance and the periodic hemodynamic signal; and, based on the level of correlation, ascertain a level of contact between the at least one intrarenal electrode and the wall of the renal artery. For some applications, a user interface is configured to output the level of contact. The periodic hemodynamic signal and the at least one time-varying component of the electrode-tissue impedance may correlate because of local mechanical changes in the blood vessel wall caused by periodic variations in blood pressure.

For some applications, the periodic hemodynamic signal is blood pressure of the subject intravascular blood pressure, or an external measurement of blood pressure). For some applications, the control circuitry is configured to calculate the level of correlation between the at least one time-varying component of the electrode-tissue impedance and the periodic hemodynamic signal by analyzing a phase difference between the at least one time-varying component of the electrode-tissue impedance and the periodic hemodynamic signal.

For some applications, the at least one time-varying component of the electrode-tissue impedance is selected from one of the following:
an electrode-tissue interface series resistance,
an elect rode-tissue interface capacitance, or
a relationship (e.g., a ratio) between the electrode-tissue impedance and the electrode-tissue interface capacitance, e.g., the quotient of (a) the electrode-tissue interface series resistance divided by (b) the electrode-tissue interface capacitance (in general, when better contact is achieved, the electrode-tissue interface series resistance increases and the electrode-tissue interface capacitance decreases; the ratio thus increases with better contact).

In some applications of the present invention, two or more sensing electrodes are disposed in or on a body of the subject, including disposing at least one of the sensing electrodes on an external surface of skin of the subject, the sensing electrodes separate and distinct from the intrarenal current-application electrodes. The control circuitry is configured to apply an electrical current between a pair of current-application electrodes, including at least one intrarenal current-application electrode; while applying the current, sense an electrical signal between two or more sensing electrodes, including the at least one external electrode; and, based on a property of the electrical signal, ascertain a level of contact between the at least one intrarenal current-application electrode and the wall of the renal artery. For some applications, the user interface is configured to output the level of contact.

For some applications, the at least one external electrode comprises at least two external electrodes. For some applications, the external electrodes are conventional electrocardiogram (ECG) electrodes, which may be positioned at one or more of the conventional ECG electrode locations on the body, and which may also be used to sense an ECG of the ablest.

Application of the electrical current may “confuse” an ECG monitor, which senses the applied signals and interprets the applied signals as drastic increases in heart rates. Effective application of the current (whether ablation or stimulation) results in stable interference with the ECG. In some applications of the present invention, such stable interference is interpreted as an indication of good contact between the at least one intrarenal electrode and tissue of the wall of the renal artery.

For some applications, the control circuitry is configured to ascertain the level of contact based on a shape of a time-varying signal rate while a current is applied. For some applications, the control circuitry is configured to ascertain the level of contact based on a stability of the time-varying signal rate. For some applications, the control circuitry is configured to extract at least one plateau from a graph of the time-varying signal rate, ascertain the shape of plateau, and ascertain the level of contact based on the shape of plateau. For some applications, the control circuitry is configured to calculate a flatness of the plateau, and ascertain the level of contact based on the flatness of the plateau.

There is therefore provided, in accordance with an application of the present invention, a method including:

disposing at least one pair of electrodes in or on a body of a subject, including disposing at least one intrarenal electrode of the pair in a renal artery of the subject;

activating control circuitry to:
(a) apply electrical pulses between the pair of electrodes,
(b) calculate at least one time-varying component of electrode-tissue impedance based on applying the pulses,
(c) sense a periodic hemodynamic signal of the subject,
(d) calculate a level of correlation between the at least one time-varying component of the electrode-tissue impedance and the periodic hemodynamic signal, and
(e) based on the level of correlation, ascertain a level of contact between the at least one intrarenal electrode and a wall of the renal artery; and

in response to the level of contact being less than a threshold level of contact, adjusting a disposition of the at least one intrarenal electrode in the renal artery.

For some applications, disposing the pair of electrodes includes disposing two intrarenal electrodes of the pair in the renal artery. For some applications, disposing the pair of electrodes includes disposing an intracorporeal reference electrode of the pair in the renal artery not in contact with the wall of the renal artery. For some applications, disposing the pair of electrodes includes disposing an external ground electrode of the pair on an external surface of the body of the subject.

For some applications:

activating the control circuitry to calculate the at least one time-varying component of the electrode-tissue impedance includes activating the control circuitry to calculate the electrode-tissue impedance, and

activating the control circuitry to calculate the level of correlation includes activating the control circuitry to calculate the level of correlation between the electrode-tissue impedance and the periodic hemodynamic signal.

For some applications:

the at least one time-varying component of the electrode-tissue impedance includes an electrode-tissue interface series resistance,

activating the control circuitry to calculate the at least one time-varying component of the electrode-tissue impedance includes activating the control circuitry to calculate the electrode-tissue interface series resistance, and

activating the control circuitry to calculate the level of correlation includes activating the control circuitry to calculate the level of correlation between the electrode-tissue interface series resistance and the periodic hemodynamic signal.

For some applications:

the at least one time-varying component of the electrode-tissue impedance includes the electrode-tissue interface series resistance and an electrode-tissue interface capacitance,

activating the control circuitry to calculate the at least one time-varying component of the electrode-tissue impedance includes activating the control circuitry to calculate the electrode-tissue impedance and the electrode-tissue interface capacitance, and a relationship between the electrode-tissue impedance and the electrode-tissue interface capacitance,

activating the control circuitry to calculate the level of correlation includes activating the control circuitry to calculate the level of correlation between (a) the relationship between the electrode-tissue impedance and the elect rode-tissue interface capacitance and (b) the periodic hemodynamic signal.

For some applications:

the at least one time-varying component of the electrode-tissue impedance includes an electrode-tisane interface capacitance,

activating the control circuitry to calculate the at least one time-varying component of the electrode-tissue impedance includes activating the control circuitry to calculate the electrode-tissue interface capacitance, and

activating the control circuitry to calculate the level of correlation includes activating the control circuitry to calculate the level of correlation between the electrode-tissue interface capacitance and the periodic hemodynamic signal.

For some applications, activating the control circuitry to calculate the level of correlation between the at least one time-varying component of the electrode-tissue impedance and the periodic hemodynamic signal includes activating the control circuitry to analyze a phase difference between the at least one time-varying component of the electrode-tissue impedance and the periodic hemodynamic signal.

For some applications, activating the control circuitry to calculate the level of correlation between the at least one time-varying component of the electrode-tissue impedance and the periodic hemodynamic signal includes activating the control circuitry to compare (a) a frequency of the at least one time-varying component of the electrode-tissue impedance and (b) a frequency of the periodic hemodynamic signal.

For some applications, activating the control circuitry to calculate the level of correlation between the at least one time-varying component of the electrode-tissue impedance and the periodic hemodynamic signal includes activating the control circuitry to calculate the level of correlation in the time domain.

For some applications, activating the control circuitry to calculate the level of correlation between the at least one time-varying component of the electrode-tissue impedance and the periodic hemodynamic signal includes activating the control circuitry to calculate the level of correlation in the frequency domain.

For some applications, activating the control circuitry to calculate the level of correlation includes activating the control circuitry to calculate the level of correlation by:

identifying a time-varying frequency component of the at least one time-varying component of the electrode-tissue impedance, and

calculating a level of correlation between the time-varying frequency component and a time-varying frequency component of the periodic hemodynamic signal.

For some applications, activating the control circuitry to calculate the level of correlation includes activating the control circuitry to compare a rate of occurrence of a feature of the time-varying frequency component with a rate of a feature of the time-varying frequency component of the periodic hemodynamic signal.

For some applications, activating the control circuitry to sense the periodic hemodynamic signal includes activating the control circuitry to sense blood pressure of the subject. For some applications, activating the control circuitry to sense the blood-pressure includes activating the control circuitry to sense intravascular blood pressure. For some applications, activating the control circuitry to sense the blood pressure includes activating the control circuitry to mechanically sense the blood pressure.

For some applications, activating the control circuitry to sense the hemodynamic signal including activating the control circuitry to sense heart beats of the subject.

For some applications:

the at least one intrarenal electrode is fixed to an elongate shaft,

disposing the at least one intrarenal electrode includes advancing the elongate shaft within the renal artery, and

activating the control circuitry to sense the hemodynamic signal includes activating the control circuitry to sense the hemodynamic signal using a sensor fixed to the elongate shaft.

For some applications, the method further includes in response to the level of contact being at least the threshold level of contact, activating the at least one intrarenal electrode to apply an excitatory current to a renal nerve of the subject. For some applications, the method further includes in response to the level of contact being at least the threshold level of contact, activating the at least one intrarenal electrode to ablate a renal nerve of the subject.

There is further provided, in accordance with an application of the present invention, a method including:

disposing at least one pair of current-application electrodes in or on a body of a subject, including disposing at least one intrarenal current-application electrode of the pair in a renal artery of the subject;

disposing two or more sensing electrodes in or on the body of the subject, including disposing at least one of the sensing electrodes on an external surface of skin of the subject, the sensing electrodes separate and distinct from the at least one intrarenal current-application electrode;

activating control circuitry to:
(a) apply an electrical current between the pair of current-application electrodes,
(b) while applying the electrical current, sense an electrical signal between the two or more sensing electrodes, including the at least one of the sensing electrodes disposed on the external surface of the skin, and
(c) based on the electrical signal, ascertain a level of contact between the at least one intrarenal current-application electrode and a wall of the renal artery; and

in response to the level of contact being less than a threshold level of contact, adjusting a disposition of the at least one intrarenal current-application electrode in the renal artery.

For some applications, disposing the two or more sensing electrodes in or on the body of the subject includes disposing at least two of the sensing electrodes on the external surface of the skin, and activating the control circuitry includes activating the control circuitry to sense the electrical signal between the at least two of the sensing electrodes disposed on the external surface of the skin.

For some applications, disposing the pair of current-application electrodes includes disposing two intrarenal current-application electrodes of the pair in the renal artery. For some applications, disposing the pair of current-application electrodes includes disposing an intracorporeal reference electrode of the pair in the renal artery not in contact oath the wall of the renal artery. For some applications, disposing the pair of current-application electrodes includes disposing an external ground electrode of the pair on an external surface of the body of the subject.

For some applications, activating the control circuitry includes activating the control circuitry to ascertain the level of contact based on a shape of the electrical signal.

For some applications, activating the control circuitry includes activating the control circuitry to:

extract at least one plateau from the electrical signal,

ascertain the shape of the plateau, and

ascertain the level of contact based on the shape of the plateau.

For some applications, activating the control circuitry includes activating the control circuitry to:

calculate e flatness of the plateau, and

ascertain the level of contact based on the flatness of the plateau.

For some applications, activating the control circuitry includes activating the control circuitry to derive from the electrical signal a time-varying signal rate over time, and to ascertain the level of contact based on the time-varying signal rate.

For some applications, activating the control circuitry includes activating the control circuitry to, while not applying the electrical current:

sense the electrical signal between the two or more sensing electrodes, including the at least one of the sensing electrodes disposed on the external surface of the skin, and

ascertain a heart rate of the subject from the electrical signal.

For some applications, activating the control circuitry includes activating the control circuitry to ascertain the level of contact based on a stability of the time-varying signal rate.

For some applications, activating the control circuitry includes activating the control circuitry to:

extract at least one plateau from a graph of the time-varying signal rate,

ascertain a shape of the plateau, and

ascertain the level of contact based on the shape of the plateau.

For some applications, activating the control circuitry includes activating the control circuitry to:

calculate a flatness of the plateau, and

ascertain the level of contact based on the flatness of the plateau.

For some applications, activating the control circuitry includes activating the control circuitry to ascertain the level of contact not responsively to an analysis of an amplitude of the electrical signal.

For some applications, the method further includes activating the control circuitry to sense a cardiac parameter of the subject between the two or more sensing electrodes, including the at least one of the sensing electrodes disposed on the external surface of the skin.

For some applications:

activating the control circuitry to apply she electrical current includes activating the control circuitry to apply an excitatory electrical current to a renal nerve of the subject, and

activating the control circuitry includes activating the control circuitry to ascertain the level of contact between the at least one intrarenal current-application electrode and the wall of the renal artery based additionally on the sensed cardiac parameter.

For some applications:

activating the control circuitry to apply the electrical current includes activating the control circuitry to apply an excitatory electrical current to a renal nerve of the subject, and

the method further includes:
activating the control circuitry to ascertain a level of suitability of a location of the at least one intrarenal current-application electrode along the renal artery based on the sensed cardiac parameter; and
based on the level of suitability being less than a threshold level of suitability, repositioning the at least one intrarenal current-application electrode along the renal artery,

For some applications:

activating the control circuitry to apply the electrical current includes activating the control circuitry to apply an excitatory electrical current to a renal nerve of the subject, and

the method further includes:
activating the control circuitry to ascertain whether the subject is a suitable candidate for renal nerve ablation based on the sensed cardiac parameter; and
in response to ascertaining that the subject is a suitable candidate, activating the at least one pair of current-application electrodes to ablate a renal nerve of the subject.

For some applications, activating the control circuitry to apply the electrical current includes activating the control circuitry to apply en excitatory electrical current to a renal nerve of the subject.

For some applications, activating the control circuitry to apply the electrical current includes activating the control circuitry to apply the electrical current with a strength sufficient to ablate a renal nerve of the subject.

There is still further provided, in accordance with an application of the present invention, a method including:

disposing a plurality of electrodes in or on a body of a subject, including disposing two or snore intrarenal electrodes of the plurality of electrodes in a renal artery of the subject;

activating control circuitry to:
(a) apply an electrical current between a pair of the electrodes, including at least a first one of the intrarenal electrodes,
(b) while applying the electrical current, sense an electrocardiogram (ECG) signal using the plurality of electrodes, including at least a second one of the intrarenal electrodes,
(c) evaluate a level of quality of the ECG signal, and
(d) based on the level of quality, ascertain a level of contact between the at least one intrarenal electrode and a wall of the renal artery; and

in response to the level of contact being less than a threshold level of contact, adjusting a disposition of the at least one intrarenal electrode in the renal artery.

For some applications:

disposing the plurality of electrodes includes disposing at least one external electrode of the plurality of electrodes on an external surface of skin of the subject, and

activating the control circuitry includes activating the control circuitry to sense the ECG signal using the plurality of electrodes, including the at least one intrarenal electrode and the at least one external electrode.

For some applications, the pair of the electrodes includes the at least a first one of the intrarenal electrodes and at least a third one of the intrarenal electrodes, and activating the control circuitry includes activating the control circuitry to apply the electrical current between the pair of intrarenal electrodes.

For some applications, disposing the plurality of electrodes includes disposing an intracorporeal reference electrode in the renal artery not in contact with the wall, of the renal artery, and activating the control circuitry includes activating the control circuitry to sense the ECG signal using the at least a second one of the intrarenal electrodes and the intracorporeal electrode.

For some applications, disposing the plurality of electrodes includes disposing an external ground electrode on an external surface of the body of the subject, and activating the control circuitry includes activating the control circuitry to sense the ECG signal using the at least a second one of the intrarenal electrodes and the external ground electrode.

For some applications, activating the control circuitry includes activating the control circuitry to derive from the ECG signal a time-varying signal rate over time, and to ascertain the level of contact based on the time-varying signal rate.

For some applications, activating the control circuitry includes activating the control circuitry to, while not applying the electrical current:

sense the ECG signal using the plurality of electrodes, including the at least a second one of the intrarenal electrodes, and

ascertain a heart rate of the subject from the electrical signal.

For some applications, activating the control circuitry includes activating the control, circuitry to ascertain the level of contact based on a stability of the time-varying signal rate.

For some applications, activating the control circuitry includes activating the control circuitry to ascertain the level of contact based on a shape of the time-varying signal rate.

For some applications, activating the control circuitry includes activating the control circuitry to:

extract at least one plateau from a graph of the time-varying signal rate.

ascertain the shape of the plateau, and

ascertain the level of contact based on the shape of the plateau.

For some applications, activating the control circuitry includes activating the control circuitry to:

calculate a flatness of the plateau, and

ascertain the level of contact based on the flatness of the plateau.

For some applications, activating the control circuitry includes activating the control circuitry to ascertain the level of contact based on a shape of the ECG signal.

For some applications, activating the control circuitry includes activating the control circuitry to:

extract at least one plateau from a graph of the ECG signal,

ascertain the shape of the plateau, and

ascertain the level of contact based on the shape of the plateau.

For some applications, activating the control circuitry includes activating the control circuitry to:

calculate a flatness of the plateau, and

ascertain the level of contact based on the flatness of the plateau.

For some applications, activating the control circuitry to apply the electrical current includes activating the control circuitry to apply an excitatory electrical current to a renal nerve of the subject. For some applications, activating the control circuitry to apply the electrical current includes activating the control circuitry to apply the electrical current with a strength sufficient to ablate a renal nerve of the subject.

There is additionally provided, in accordance with an application of the present invention, a method, including:

disposing a plurality of electrodes in or on a body of a subject, including disposing at least one intrarenal electrode of the plurality of electrodes in a renal artery of the subject;

activating control circuitry to:
(a) apply an electrical current between a pair of the electrodes, including at least one of the intrarenal electrodes,
(b) while applying the electrical current, sense an electrocardiogram (ECG) signal using the plurality of electrodes,
(c) derive a time-varying signal rate from the ECG signal, and
(d) based on a stability of the time-varying signal rate, ascertain a level of contact between the at least one intrarenal electrode and a wall of the renal artery; and

in response to the level of contact being less than a threshold level of contact, adjusting a disposition of the at least one intrarenal electrode in the renal artery.

For some applications, the at least one of the intrarenal electrodes is at least a first one of the intrarenal electrodes, and activating the control circuitry includes activating the control circuitry to sense the electrocardiogram (ECG) signal using the plurality of electrodes, including at least a second one of the intrarenal electrodes.

For some applications:

disposing the plurality of electrodes includes disposing at least one external electrode of the plurality of electrodes on an external surface of skin of the subject, and

activating the control circuitry includes activating the control circuitry to sense she ECG signal using the plurality of electrodes, including the at least one external electrode.

For some applications, activating the control circuitry includes activating the control circuitry to, while not applying the electrical current:

sense the ECG signal using the plurality of electrodes,

derive the time-varying signal rate from the ECG signal,

ascertain a heart rate of the subject from the time(tm) varying signal rate.

For some applications, activating the control circuitry to apply the electrical current includes activating the control circuitry to apply an excitatory electrical current to a renal nerve of the subject. For some applications, activating the control circuitry to apply the electrical current includes activating the control circuitry to apply the electrical current with a strength sufficient to ablate a renal nerve of the subject.

There is yet additionally provided, in accordance with an application of the present invention, apparatus including:

at least one pair of electrodes, which include at least one intrarenal electrode configured to be disposed in a renal artery of a subject;

control circuitry, configured to:
(a) apply electrical pulses between the pair of electrodes,
(b) calculate at least one time-varying component of electrode-tissue impedance based on applying the pulses,
(c) sense a periodic hemodynamic signal of the subject,
(d) calculate a level of correlation between the at least one time-varying component of the electrode-tissue impedance and the periodic hemodynamic signal, and
(e) based on the level of correlation, ascertain a level of contact between the at least one intrarenal electrode and a wall of the renal artery; and

a user interface, which is configured to output the level of contact.

For some applications, the pair of electrodes includes two intrarenal electrodes configured to be disposed in the renal artery. For some applications, the pair of electrodes includes an intracorporeal reference electrode configured to be disposed in the renal artery not in contact with the wall of the renal artery. For some applications, the pair of electrodes includes an external ground electrode configured to be disposed on an external surface of a body of the subject.

For some applications, the control, circuitry is configured to:

calculate the at least one time-varying component of the electrode-tissue impedance by calculating the electrode-tissue impedance, and

calculate the level of correlation between the electrode-tissue impedance and the periodic hemodynamic signal.

For some applications, the at least one time-varying component of the electrode-tisane impedance includes an electrode-tissue interface series resistance, and the control circuitry is configured to:

calculate the at least one time-varying component of the electrode-tissue impedance by calculating the electrode-tissue interface series resistance, and

calculate the level of correlation between the electrode-tissue interface series resistance and the periodic hemodynamic signal.

For some applications, the at least one time-varying component of the electrode-tissue impedance includes the electrode-tissue interface series resistance and an electrode-tissue interface capacitance, and the control circuitry is configured to:

calculate the at least one time-varying component of the electrode-tissue impedance by calculating the electrode-tissue impedance and the electrode-tissue interface capacitance, and a relationship between the electrode-tissue impedance and the electrode-tissue interface capacitance,

calculate the level of correlation between (a) the relationship between the electrode-tissue impedance and the electrode-tissue interface capacitance and (b) the periodic hemodynamic signal.

For some applications, the at least one time-varying component of the electrode-tissue impedance includes an electrode-tissue interface capacitance, and the control circuitry is configured to:

calculate the at least one time-varying component of the electrode-tissue impedance by calculating the electrode-tissue interface capacitance, and

calculate the level of correlation between the electrode-tissue interface capacitance and the periodic hemodynamic signal.

For some applications, the control circuitry is configured to calculate the level of correlation between the at least one time-varying component of the electrode-tissue impedance and the periodic hemodynamic signal by analyzing a phase difference between the at least one time-varying component of the electrode-tissue impedance and the periodic hemodynamic signal.

For some applications, the control circuitry is configured to calculate the level of correlation between the at least one time-varying component of the electrode-tissue impedance and the periodic hemodynamic signal by comparing (a) a frequency of the at least one time-varying component of the electrode-tissue impedance and (b) a frequency of the periodic hemodynamic signal.

For some applications, the control circuitry is configured to calculate the level of correlation between the at least one time-varying component of the electrode-tissue impedance and the periodic hemodynamic signal by calculating the level of correlation in the time domain.

For some applications, the control circuitry is configured to calculate the level of correlation between the at least one time-varying component of the electrode-tissue impedance and the periodic hemodynamic signal by calculating the level of correlation in the frequency domain.

For some applications, the control circuitry is configured to calculate the level of correlation by:

identifying a time-varying frequency component of the at least one time-varying component of the electrode-tissue impedance, and

calculating a level of correlation between the time-varying frequency component and a time-varying frequency component of the periodic hemodynamic signal.

For sore applications, the control circuitry is configured to calculate the level of correlation by comparing a rate of occurrence of a feature of the time-varying frequency component with a rate of a feature of the time-varying frequency component of the periodic hemodynamic signal.

For some applications, the periodic hemodynamic signal is blood pressure of the subject, and the control circuitry is configured to sense the blood pressure. For some applications, the blood pressure is intravascular blood pressure, and the control circuitry is configured to sense the intravascular blood pressure. For some applications, the apparatus further includes a mechanical sensor of the blood pressure, and the control circuitry is configured to sense the blood pressure using the mechanical sensor.

For some applications, the hemodynamic signal includes heart beats of the subject, and the control circuitry is configured to sense the heart beats.

For some applications, the apparatus further includes:

an elongate shaft, to which the at least one intrarenal electrode is fixed; and

a sensor, which is fixed to the elongate shaft,

and the control circuitry is configured to sense the hemodynamic signal using the sensor.

For some applications, the control circuitry is configured to activate the at least one intrarenal electrode to apply an excitatory current to a renal nerve of the subject, in response to the level of contact being at least a threshold level of contact. For some applications, the control circuitry is configured to activate the at least one intrarenal electrode to ablate a renal nerve of the subject, in response to the level of contact being at least a threshold level of contact.

There is also provided, in accordance with an application of the present invention, apparatus including:

at least one pair of current-application electrodes, which include at least one intrarenal electrode configured to be disposed in a renal artery of a subject;

two or more sensing electrodes, which are separate and distinct from the intrarenal current-application electrodes, and which are configured to be disposed in or on a body of the subject, wherein the two or more sensing electrodes include at least one external electrode, which is configured to be disposed on an external surface of skin of the subject;

control circuitry, configured to:
(a) apply an electrical current between the pair of current-application electrodes,
(b) while applying the electrical current, sense an electrical signal between the two or more sensing electrodes, including the at least one external electrode, and
(e) based on a property of the electrical signal, ascertain a level of contact between the at least one Intrarenal current-application electrode and a wall of the renal artery; and

a user interface, which is configured to output the level of contact.

For some applications, the at least one external electrode includes at least two external electrodes, and the control circuitry is configured to sense the electrical signal between the at least two external electrodes. For some applications, the pair of current-application electrodes includes two intrarenal current-application electrodes configured to be disposed in the renal artery. For some applications, the pair of current-application electrodes includes an intracorporeal reference electrode configured to be disposed in the renal artery not in contact with the wall of the renal artery. For some applications, the pair of current-application electrodes includes an external ground electrode configured to be disposed on an external surface of the body of the subject.

For some applications, the control circuitry is configured to ascertain the level of contact based on a shape of the electrical signal.

For some applications, the control circuitry is configured to:

extract at least one plateau from the electrical signal,

ascertain the shape of the plateau, and

ascertain the level of contact based on the shape of the plateau.

For some applications, the control circuitry is configured to:

calculate a flatness of the plateau, and

ascertain the level of contact based on the flatness of the plateau.

For some applications, the control circuitry is configured to derive from the electrical signal a time-varying signal rate over time, and to ascertain the level of contact based on the time-varying signal rate.

For some applications, the control circuitry is configured to, while not applying the electrical current:

sense the electrical signal between the two or more sensing electrodes, including the two or more sensing electrodes, including the at least one external electrode, and

ascertain a heart rate of the subject from the electrical signal.

For some applications, the control circuitry is configured to ascertain the level of contact based on a stability of the time-varying signal rate.

For some applications, the control circuitry is configured to:

extract at least one plateau from a graph of the time-varying signal rate,

ascertain a shape of the plateau, and

ascertain the level of contact based on the shape of the plateau.

For some applications, the control circuitry is configured to:

calculate a flatness of the plateau, and

ascertain the level of contact based on the flatness of the plateau.

For sortie applications, the control circuitry is configured to ascertain the level of contact not responsively to an analysis of an amplitude of the electrical signal.

For some applications, the control circuitry is configured to sense a cardiac parameter of the subject between the two or more sensing electrodes, including the at least one external electrode.

For some applications, the control circuitry is configured to:

apply the electrical current as an excitatory electrical current to a renal nerve of the subject, and

ascertain the level of contact between the at least one intrarenal current-application electrode and the wall of the renal artery based additionally on the sensed cardiac parameter.

For some applications:

the control circuitry is configured to (a) apply the electrical current as an excitatory electrical current to a renal nerve of the subject, and (b) ascertain a level of suitability of a location of the at least one intrarenal current-application electrode along the renal artery based on the sensed cardiac parameter, and

the user interface is configured to output information based on the level of suitability.

For some applications:

the control circuitry is configured to (a) apply the electrical current as an excitatory electrical current to a renal nerve of the subject, and (b) ascertain whether the subject is a suitable candidate for renal nerve ablation based on the sensed cardiac parameter,

the user interface is configured to output an indication regarding whether the subject is a suitable candidate.

For some applications, the control circuitry is configured to:

apply the electrical current as an excitatory electrical current to a renal nerve of the subject,

ascertain whether the subject is a suitable candidate for renal nerve ablation based on the sensed cardiac parameter, and

activate the at least one pair of current-application electrodes to ablate a renal nerve of the subject, in response to ascertaining that the subject is a suitable candidate.

For some applications, the control circuitry is configured to apply the electrical current as an excitatory electrical current. For some applications, the control circuitry is configured to apply the electrical current with a strength sufficient to ablate a renal nerve of the subject.

There is further provided, in accordance with an application of the present invention, apparatus including:

a plurality of electrodes, which include two or more intrarenal electrodes configured to be disposed in a renal artery of a subject;

control circuitry, configured to:
(a) apply an electrical current between a pair of the electrodes, including at least a first one of the intrarenal electrodes,
(b) while applying the electrical current, sense an electrocardiogram (ECG) signal using the plurality of electrodes, including at least a second one of the intrarenal electrodes,
(c) evaluate a level of quality of the ECG signal, and
(d) based on the level of qualify, ascertain a level of contact between the at least one intrarenal electrode and a wall of the renal artery; and

a user interface, which is configured to output the level of contact.

For some applications:

the plurality of electrodes includes at least one external electrode configured to be disposed on an external surface of skin of the subject, and

the control circuitry is configured to sense the ECG signal using the plurality of electrodes, including the at least one intrarenal electrode and the at least one external electrode.

For some applications, the pair of the electrodes includes the at least a first one of the intrarenal electrodes and at least a third one of the intrarenal electrodes, and the control circuitry is configured to apply the electrical current between the pair of intrarenal electrodes.

For some applications, the plurality of electrodes includes an intracorporeal reference electrode configured to be disposed in the renal artery not in contact with the wall of the renal artery, and the control circuitry is configured to sense the ECG signal using the at least a second one of the intrarenal electrodes and the intracorporeal electrode.

For some applications, the plurality of electrodes includes an external ground electrode configured to be disposed on an external surface of a body of the subject, and the control circuitry is configured to sense the ECG signal using the at least a second one of the intrarenal electrodes and the external ground electrode.

For some applications, the control circuitry is configured to derive from the ECG signal a time-varying signal rate over time, and to ascertain the level of contact based on the time-varying signal rate.

For some applications, the control circuitry is configured to, while not applying the electrical current:

sense the ECG signal using the plurality of electrodes, including the at least a second one of the intrarenal electrodes, and

ascertain a heart rata of the subject from the electrical signal.

For some applications, the control circuitry is configured to ascertain the level of contact based on a stability of the time-varying signal rate.

For some applications, the control circuitry is configured to ascertain the level of contact based on a shape of the time-varying signal rate.

For some applications, the control circuitry is configured to:

extract at least one plateau from a graph of the time-varying signal rate,

ascertain the shape of the plateau, and

ascertain the level of contact based on the shape of the plateau.

For some applications, the control circuitry is configured to:

calculate a flatness of the plateau, and

ascertain the level of contact based on the flatness of the plateau.

For some applications, the control circuitry is configured to ascertain the level of contact based on a shape of the ECG signal.

For some applications, the control circuitry is configured to:

extract at least one plateau from a graph of the ECG signal,

ascertain the shape of the plateau, and

ascertain the level of contact based on the shape of the plateau.

For some applications, the control circuitry is configured to:

calculate a flatness of the plateau, and

ascertain the level of contact based on the flatness of the plateau.

For some applications, the control circuitry is configured to apply the electrical current as an excitatory electrical current to a renal nerve of the subject. For some applications, the control circuitry is configured to apply the electrical current with a strength sufficient to ablate a renal nerve of the subject.

There is still further provided, in accordance with an application of the present invention, apparatus including:

a plurality of electrodes, which include at least one intrarenal electrode configured to be disposed in a renal artery of a subject;

control circuitry, configured to:
(a) apply an electrical current between a pair of the electrodes, including at least one of the intrarenal electrodes,
(b) while applying the electrical current, sense an electrocardiogram (ECG) signal using the plurality of electrodes,
(c) derive a time-varying signal rate from the ECG signal, and
(d) based on a stability of the time-varying signal rate, ascertain a level of contact between the at least one intrarenal electrode and a wall of the renal artery; and

a user interface, which is configured to output the level of contact.

For sees applications, the at least one of the intrarenal electrodes is at least a first one of the intrarenal electrodes, and activating the control circuitry includes activating the control circuitry to sense the electrocardiogram (ECG) signal using the plurality of electrodes, including at least a second one of the intrarenal electrodes.

For some applications:

the plurality of electrodes includes at least one external electrode configured to be disposed on an external surface of skin of the subject, and

the control circuitry is configured to sense the ECG signal using the plurality of electrodes, including the at least one external electrode.

For some applications, the control circuitry is configured to, while not applying the electrical current:

sense the ECG signal using the plurality of electrodes,

derive the time-varying signal rate from the ECG signal,

ascertain a heart rate of the subject from the time-varying signal rate.

For some applications, the control, circuitry is configured to apply an excitatory electrical current to a renal nerve of the subject. For some applications, the control circuitry is configured to apply the electrical current with a strength sufficient to ablate a renal nerve of the subject.

The present invention will be sore fully understood from the following detailed description of embodiments thereof, taken together with the drawings, in which:

### BRIEF DESCRIPTION OF THE DRAWINGS

FIG. 1 is a schematic illustration of a system for ablating and/or stimulating nerve tissue of a blood vessel of a subject, such as a renal artery, in accordance with some applications of the present invention;

FIGS. 2A-B are schematic illustrations of exemplary deployments of an electrode unit of the system of FIG. 1 in the renal artery, in accordance with an application of the present invention;

FIGS. 3A-B are graphs of respective electrode-tissue interface series resistance signals, and respective intravascular blood pressure signals, measured in accordance with an application of the present invention;

FIG. 3C is a graph of an intravascular blood pressure signal and of a contact index, as determined in accordance with an application of the present invention;

FIG. 3D shows a graph of the frequency domain of the intravascular blood pressure signal shown in FIG. 3C and a graph of the frequency domain of the contact index shown in FIG. 3C, in accordance with an application of the present invention;

FIG. 4 is a flow chart that schematically illustrated a method for ascertaining a level of contact between at least one intrarenal electrode and a wall of the renal artery, in accordance with an application of the present invention;

FIG. 5 is a schematic illustration of another system for ablating and/or stimulating nerve tissue of a blood vessel of a subject, in accordance with an application of the present invention;

FIGS. 6A-B are graphs of signal rate values, calculated in accordance with an application of the present invention; and

FIG. 7 is a flow chart that schematically illustrated another method for ascertaining a level of contact between at least one intrarenal electrode and a wall of the renal artery, in accordance with an application of the present invention.

### DETAILED DESCRIPTION OF APPLICATIONS

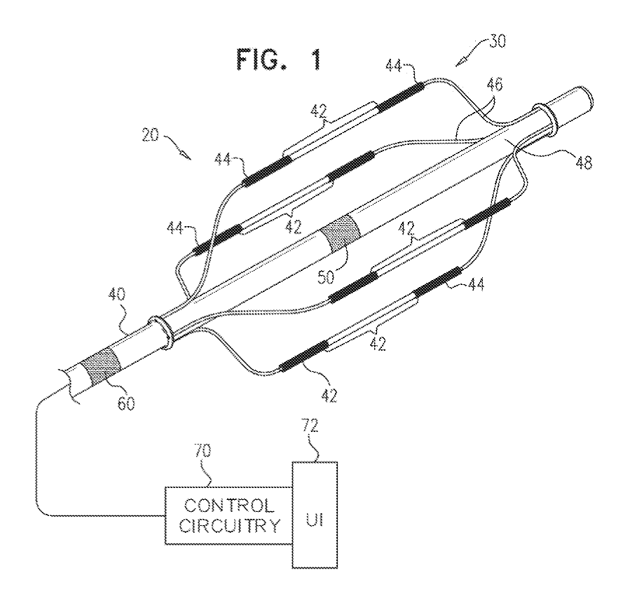

FIG. 1 is a schematic Illustration of a system 20 for ablating and/or stimulating nerve tissue of a blood vessel of a subject, such as a renal artery, in accordance with some applications of the present invention. System 20 typically comprises an electrode unit 30, an elongate shaft 40, control circuitry 70, and a user interface 72. For some applications, system 20 is configured to stimulate, sense, and/or ablate the nerve tissue of the blood vessel. For some applications, system 20 is configured to apply stimulation energy to the blood vessel wall, and, optionally, to sense a resulting signal for purposes of sensing contact between electrodes and the wall of the renal artery. Alternatively or additionally, for some applications, system 20 is configured to apply ablation energy to the blood vessel wall (and to adjacent nerve tissue), so as to block endogenous action potentials from propagating through the nerve tissue (e.g., to ablate nerve tissue in a first portion of the nerve tissue, so as to permanently block pathogenic action potentials (afferent and/or efferent) from propagating past the first portion of the nerve tissue).

Electrode unit 30 comprises at least one pair 42 of intrarenal electrodes 44, which is typically configured to be disposed in a renal artery of the subject. For example, in the configuration shown in FIG. 1, electrode unit 30 comprises four pairs 42 of intrarenal electrodes 44, Electrode unit 30 typically further comprises one or more support struts 46, to which intrarenal electrodes 44 are fixed. For example, each pair 42 of intrarenal electrodes 44 may be fixed to a separate support strut 46. For example, support struts 46 may be elastically-bendable wires, flat strips, or pieces of metal laser out from a tube, e.g., comprising a polymer such as nylon and/or a metal such as Nitinol, biased to assume the open state.

For some applications, support struts 46 are arranged as a basket, which is configured to assume open (radially-expanded) and closed (radially-compressed) states. For example, one end of the support struts may slide along a central shaft 48 of electrode unit 30 in order to transition between the open and closed states. The electrode unit is advanced through the vasculature in a closed state, and transitioned to the open state once positioned at a desired target location in the renal artery. Electrode unit 30 may, for example, implement techniques described in International Application PCT/IB2015/053350, filed May 7, 2015, which is assigned to the assignee of the present application and is incorporated herein by reference.

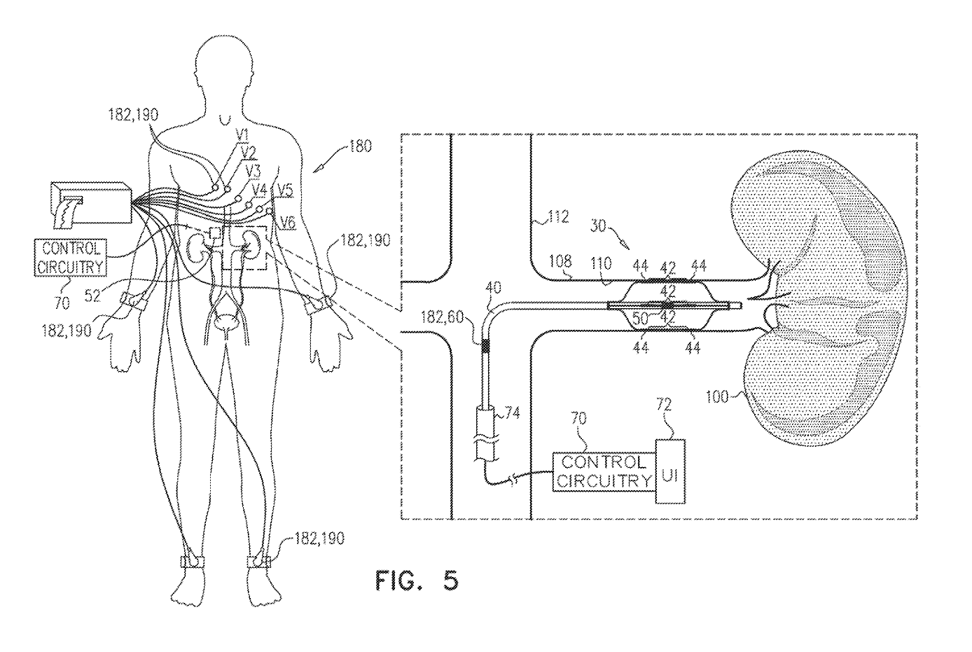

For some applications, system 20 further comprises an intracorporeal reference electrode 50, which is configured to be disposed in the renal artery not in contact with the wall of the renal artery, intracorporeal reference electrode 50 may be positioned, for example, on central shaft 48 of electrode unit 30 (e.g., as shown, or elsewhere, such as at a distal tip of electrode unit 30), or on elongate shaft 40 (e.g., similar to sensor 60, as described below). Alternatively or additionally, for some applications, system 20 further comprises an external ground electrode 52 (e.g., a ground pad), such as shown, for example, in FIG. 5, which is typically positioned in contact with an external surface of the subject's skin, such as on the back.

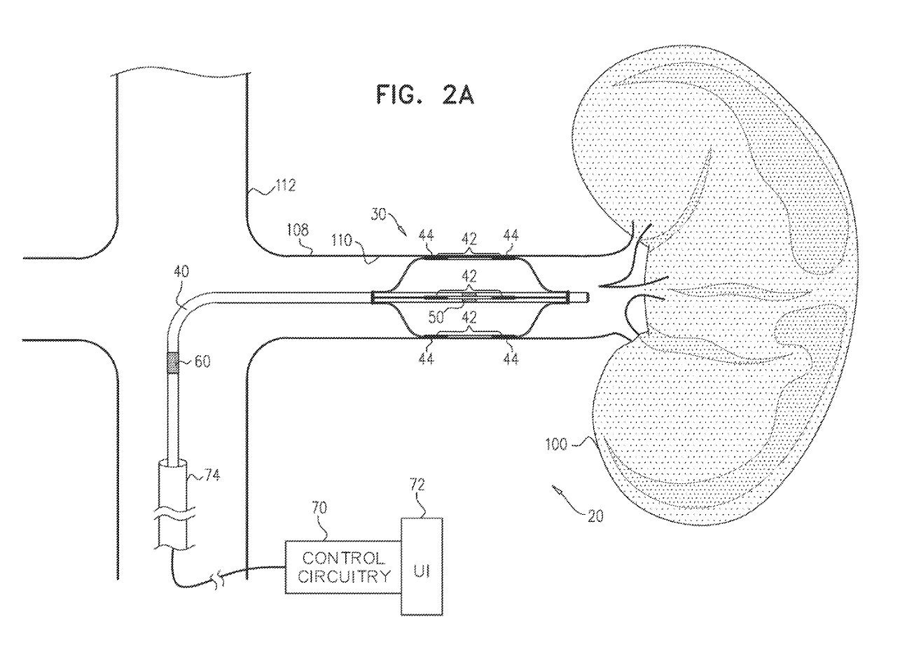

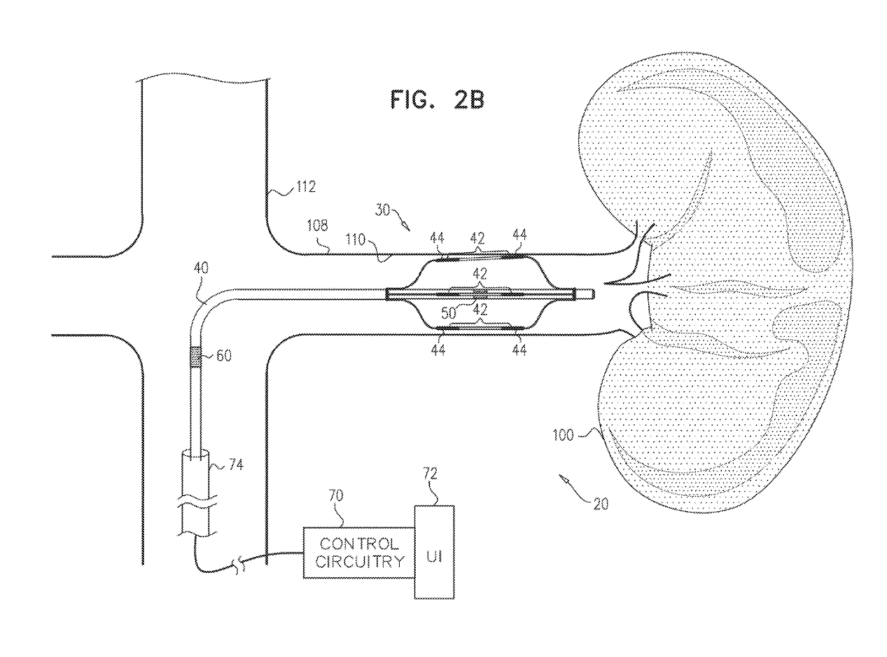

Reference is now made to FIGS. 2A-B, which are schematic illustrations of exemplary deployments of electrode unit 30 in a renal artery 108, in accordance with an application of the present invention. Elongate shaft 40 and electrode unit 30 are typically advanced in a minimally-invasive percutaneous procedure, typically through a sheath 74. During activation of electrode unit 30 for ablating nerve tissue, as described above, it is important that there be good contact between intrarenal electrodes 71 and a wall 119 of renal artery 108. FIG. 2A schematically illustrates potential good contact, while FIG. 2B schematically illustrates poor contact. It is noted that although FIG. 2A illustrates good mechanical contact, in some cases such good mechanical contact does not result in high quality electrical contact (for example, the level of force with which the electrodes touch the well stay be insufficient to provide good electrical contact, and/or the microscopic interface between the electrode material and the tissue of the wall may be poor). In some applications of the present invention, a level of contact between at least one intrarenal electrode 44 and wall 110 of renal artery 108 is ascertained. If the level is not sufficient, a disposition of one or more of intrarenal electrodes 44 is typically adjusted. For some applications, the contact of each of intrarenal electrodes 44 is separately ascertained, or of each pair 42 of intrarenal electrodes 44, and, optionally, separately outputted to the user of the system. For some applications, the contact level of each of intrarenal electrodes 44 is separately ascertained. Alternatively, the contact level of pairs 42 of intrarenal electrodes 44 is separately ascertained. Further alternatively, the contact level of intrarenal electrodes 44 in more than one pair is simultaneously ascertained.

In some applications or the present invention, control circuitry 70 is configured to:
apply electrical pulses between a pair of electrodes, such as between (a) pair 42 of intrarenal electrodes 44, (b) one of intrarenal electrodes 44 and intracorporeal reference electrode 50, or (c) one of intrarenal electrodes 44 and external ground electrode 52,
calculate at least one time-varying component of electrode-tissue impedance based on applying the pulses,
sense a periodic hemodynamic signal of the subject,
calculate a level of correlation between the at least one time-varying component of the electrode-tissue impedance and the periodic hemodynamic signal, and
based on the level of correlation, ascertain a level of contact between the at least one intrarenal electrode 44 and wall 110 of renal artery 108.

For some applications, user interface 72 is configured to output the level of contact.

The periodic hemodynamic signal and the at least one time-varying component of the electrode-tissue impedance may correlate because of local mechanical changes in the blood vessel wall caused by periodic variations in blood pressure.

For some applications, control circuitry 70 is configured to calculate the level of correlation in a moving window, e.g., having a duration between 3 and 8 seconds, e.g., a 4 second duration.

For configurations in which the electrical pulses are applied in a unipolar mode between (a) one of intrarenal electrodes 44 and intracorporeal reference electrode 50 or (b) one of intrarenal electrodes 44 and external ground electrode 52, the wall contact and disposition of each of intrarenal electrodes 44 can be separately checked, acid each electrode separately repositioned as necessary. For some applications, the electrical pulses are applied in the unipolar mode described above and in a bipolar mode (between a pair of intrarenal electrodes 44). The unipolar and bipolar modes may be performed simultaneously, or in sequence.

For some applications, a method is provided that comprises activating control circuitry 70 to perform the above-mentioned functions. In response to the level of contact being less than a threshold level of contact, a disposition of the at least one intrarenal electrode 44 in renal artery 108 is adjusted (typically manually, by a physician performing the procedure), in an attempt to achieve a higher level of contact. Typically, the level of contact is again measured, and, if necessary, the disposition of the at least one intrarenal electrode 44 is again adjusted. These steps may be repeated as many times as necessary until sufficiently good contact is achieved. For some applications, control circuitry 70 generally constantly ascertains, and optionally outputs, the level of contact.

For some applications, the threshold level of contact is preprogrammed in control circuitry 70. Alternatively, the threshold level of contact is obtained during a procedure for each subject individually; for example, control circuitry 70 may set the threshold level of contact during deployment of electrode unit 30 in renal artery 108, or by temporarily deploying electrode unit 30 in the aorta and measuring the electrode-tissue impedance while assuring that no contact is made between intrarenal electrodes 44 and the wall of the aorta.

As mentioned above, for some applications, electrode unit 30 further comprises reference electrode 50, which may be fixed to central shaft 48. For some applications, a calibration process is performed outside the subject's body (e.g., In saline solution), or inside the subject's body (for example, in the aorta), to measure the impedance when there is no contact between intrarenal electrodes 44 and the wall of the renal artery. This calibration measurement may serve as a predefined value for the threshold that is used to ascertain the level of contact between the intrarenal electrodes and the wall of the renal artery. Alternatively or additionally, reference electrode 50 may serve as a reference electrode for checking the electrodes connectivity and quality of assembly, for example by measuring the impedance of each electrode with the reference electrode, and comparing the value of this measurement with other electrodes or with a predefined value set by the system.

For some applications, the periodic hemodynamic signal is blood pressure of the subject (e.g., intravascular blood pressure), and control circuitry 70 is configured to sense the blood pressure (e.g., using a mechanical sensor, such as a piezoelectric sensor, or an optical sensor, both as known in the art). For some applications, control circuitry 70 is configured to calculate the level of correlation between the at least one time-varying component of the electrode-tissue impedance and the periodic hemodynamic signal by analyzing a phase difference between the sit least one time-varying component of the electrode-tissue impedance and the periodic hemodynamic signal. for some

applications, control circuitry 70 is configured no define the level of correlation by analyzing a difference in a value of the at least one time-varying component of electrode-tissue impedance at systolic blood pressure and at diastolic blood pressure. By way of example and not limitation, an electrode-tissue interface series resistance and/or an electrode-tissue interface capacitance may be less at systolic blood pressure than at diastolic blood pressure.

For some applications, the hemodynamic signal includes heart beats of the subject, and control circuitry 10 is configured to sense the heart beats.

For some applications, control circuitry 70 is configured to calculate the level of correlation by (a) identifying a time-varying frequency component of the at least one time-varying component of the electrode-tissue impedance, and (b) calculating a level of correlation between the time-varying frequency component and a time-varying frequency component of the periodic hemodynamic signal. For some applications, control circuitry 70 derives the time-varying frequency component using a fast Fourier transform (FFT) of the at least one time-varying component of the electrode-tissue impedance (e.g., using the frequency with the greatest magnitude). For some applications, control circuitry 70 is configured to calculate the level of correlation by comparing a rate of occurrence of a feature of the time-varying frequency component with a rate of a feature of the time-varying frequency component of the periodic hemodynamic signal. For example, control circuitry 70 may calculate maxima frequency (above a predefined threshold) in the time domain and analyze the maxima frequency in time (for example, over 10 seconds) to ascertain a correlation with the frequency of the blood pressure peaks.

For some applications, the at least one intrarenal electrodes 44 is fixed to elongate shaft 40 (e.g., via support struts 46: and/or central shaft 48). System 20 further comprises a sensor 60, which is fixed to elongate shaft 40 or electrode unit 30 (e.g., on central shaft 48), Although sensor 60 is shown fixed to elongate shaft 40 at a particular location, the sensor may be positioned anywhere along the elongate shaft, including at more distal locations. Control circuitry 70 is configured to sense the hemodynamic signal using sensor 60.

For some applications, control circuitry 70 is configured to calculate the at least one time-varying component of electrode-tissue impedance by:
driving the at least one pair of electrodes to apply the electrical pulses; for example, the electrical pulses may be applied as biphasic, high frequency, low amplitude pulses, e.g., between 1 and 5 kHz, such as between 1.5 and 2.5 kHz (e.g., 2 kHz) rectangular biphasic pulses, with each pulse having a duration of between 150 and 2 ms, e.g., 500 us, with as low an interpulse delay as possible; the pulses are typically delivered in trains of pulses having between 10 and 200 cycles e.g., with an inter-train inactive period having a duration of 0.1 seconds and 2 seconds, e.g., 400 us (e.g., two trains may be delivered per second), and,
sensing, using each pair of electrodes, pulses generated in response to the applied electrical pulses; the pulses are typically sensed in a time window, for example having a duration or at least 0.1 ms, e.g., 50 ms; for example, the sampling rats may be between 1 and 100 kHz.

For some applications, control circuitry 70 is configured to compare the shape of the detected pulses to the delivered constant-current rectangular pulses, using a Randles circuit, as is known in the art, in order to extract the following parameters from the sensed pulses:
an electrode-tissue interface series resistance, from, the rise time of the pulse, e.g., in accordance with the following equation: R1 =V_{AMP}/I_{AMP}
a parallel electrode-medium interface resistance, and
an electrode-tissue interface capacitance, from the voltage buildup during the pulse; e.g., extracted using a linear approximation at the beginning of the pulse in accordance with the following equation: C=I_{CONST}*ΔT/ΔV,

in which:

I_{AMP}=I_{@T1}−I_{@T0}

V_{AMP}=V_{@T1s}−V_{@0us}

I_{CONST}=E(I_{@T2:T3})

ΔT=T₃−T₂,

ΔT=V_{@T3}−V_{@T2}

T₀=0us, T₁=80us,T₂100us,T₃=250us

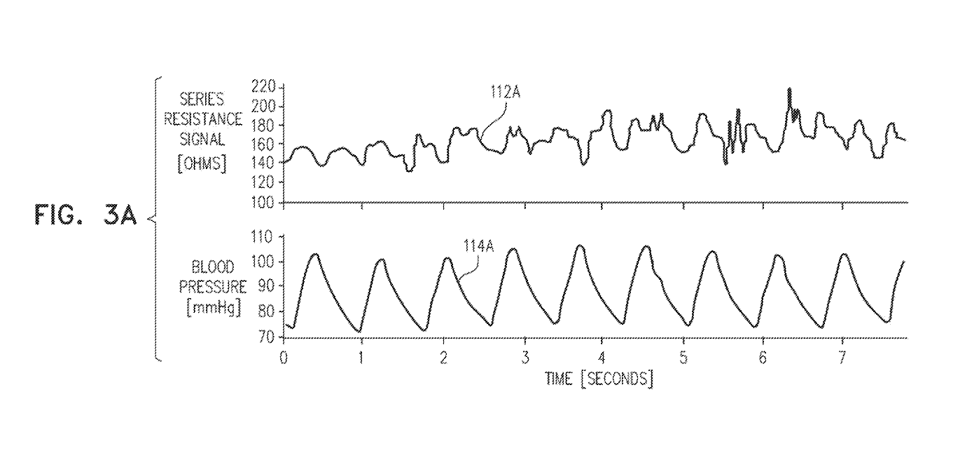

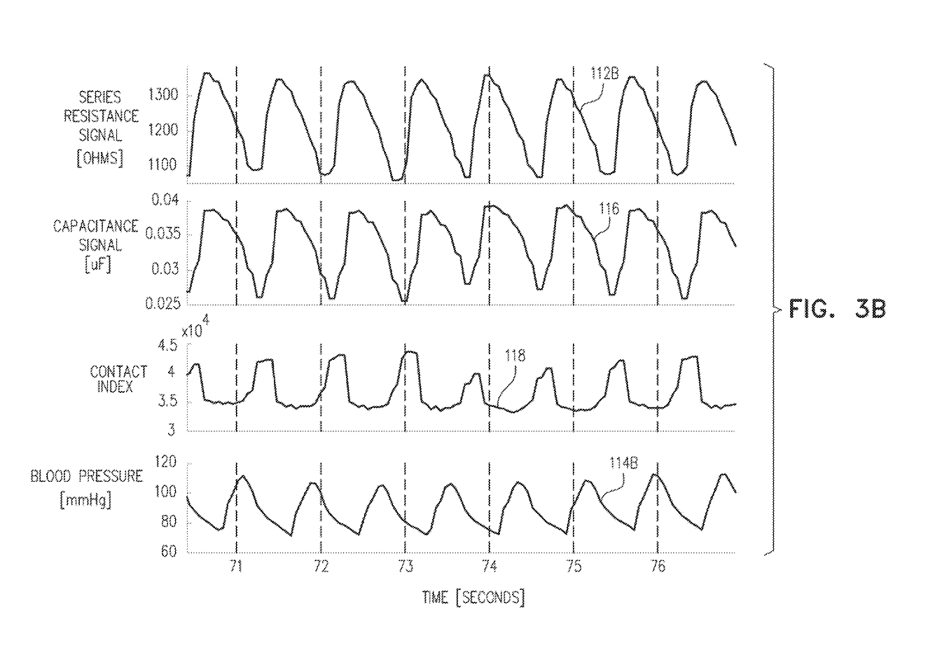

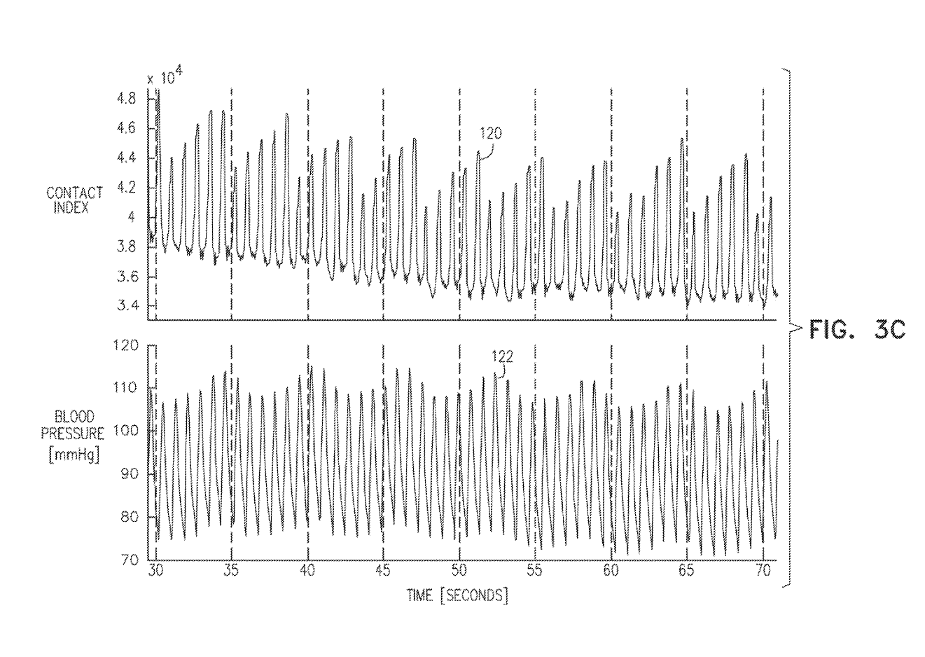

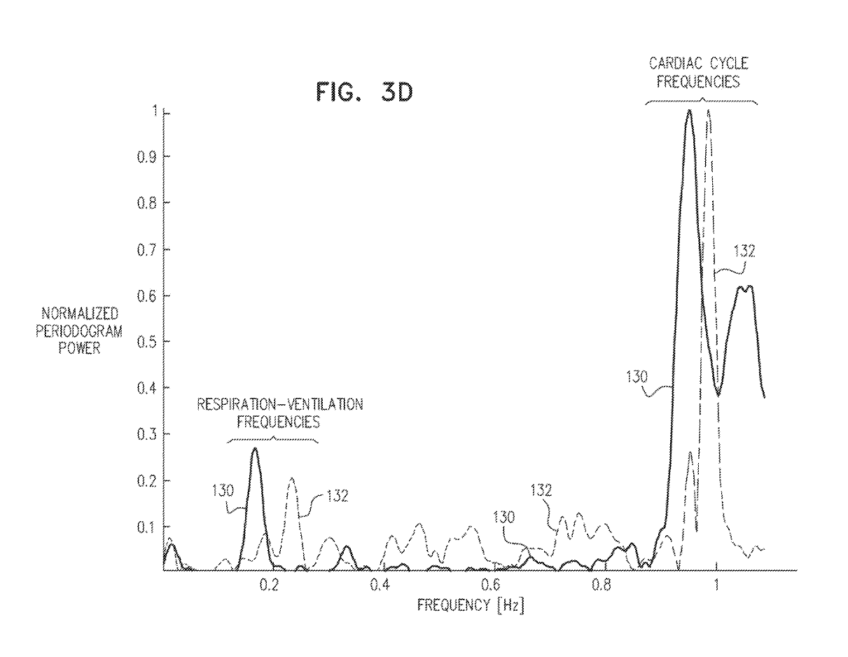

For some applications, the at least one time-varying component of the electrode-tissue impedance is selected from one of the following:
the electrode-tissue interface aeries resistance,
the electrode-tissue interface capacitance, or
a relationship (e.g., a ratio) between the electrode-tissue impedance and the electrode-tissue interface capacitance, e.g., the quotient of (a) the electrode-tissue interface series resistance divided by (b) the electrode-tissue interface capacitance (in general, when better contact is achieved, the electrode-tissue interface aeries resistance increases and the electrode-tissue interface capacitance decreases; the ratio thus increases with better contact). (This quotient is referred to as the “contact index” in FIGS. 3B-C, described hereinbelow.)

Reference is now made to FIGS. 3A-B, which are graphs of respective electrode-tissue interface series resistance signals 112A and 112B, and respective intravascular blood pressure signals 114A and 114B, measured in accordance with an application of the present invention. In addition, FIG. 3B includes graphs of an electrode-tissue interface capacitance signal 116 and a contact index 118 (described hereinabove with reference to FIGS. 2A-B). These signals were measured during in vivo experiments conducted on behalf of the inventors in two respective female pigs. Electrode-tissue interface series resistance signals 112A and 112B and electrode-tissue interface capacitance signal 116 were measured, using an electrode unit similar to intravascular device 1000 described in International Application PCT/IB2015/053350, filed May 7, 2015, which is assigned to the assignee of the present application and is incorporated herein by reference. The intravascular device used, like some configurations electrode unit 30, comprised four pairs of electrodes distributed similarly to the electrodes of electrode unit 30, and additionally comprises a balloon. Electrode-tissue interface series resistance signal 112A, shown in FIG. 3A, was measured with the electrode unit in a deflated configuration (similar to a closed basket configuration of electrode unit 30), with poor contact with the wall of the renal artery, while electrode-tissue interface series resistance signal 112B and electrode-tissue interface capacitance signal 116, shown in FIG. 3B, were measured with the electrode unit in an inflated balloon configuration (similar to an open basket configuration of electrode unit 30), with good contact with the wall or the renal artery. Blood pressure signals 114A and 114B were measured using an intravascular blood pressure sensor.

As can be seen in FIGS. 3A-B, electrode-tissue interface series resistance signal 112B correlates better with blood pressure signal 114B than electrode-tissue interface series resistance signal 112A correlates with blood pressure signal 114A, can also be seen in FIG. 3B, electrode-tissue interface capacitance signal 116 and contact index 118 both correlate well with blood pressure signal 114B. For example, good correlation between electrode-tissue interface series resistance signal 112B and blood pressure signal 114B may be reflected by similar frequencies in the signals, as described hereinbelow with reference to FIGS. 3C and 3D. Alternatively or additionally, good correlation may be reflected by a decline in electrode-tissue interface series resistance signal 112B and/or in elect rode-tissue interface capacitance signal 116 at the systolic blood pressure peaks of blood pressure signal 114B.

Although the signals shown in FIG. 3B correlate well with blood pressure, the signals may reflect some phase shift and frequency shifts, e.g., because (a) an insufficient sampling frequency (the rate of stimulation was only 20 Hz) reduced temporal resolution and enhanced processing errors, (b) local vessel wall fluctuations may have distorted frequencies because of vessel wave dynamics, and (c) the respective positions of the blood pressure sensor and the electrodes may have contributed to the phase and/or frequency shifts. (It is noted that the absolute values of the series resistance signals shown in FIG. 3A are substantially lower than those shown in FIG. 3B. These differences may have resulted from the poorer contact of the electrode with wall 110 during the measurements of FIG. 3A than during the measurements of FIG. 3B. Other differences in electrode placement may also have contributed to these differences.)

Reference is made to FIG. 3C, which is a graph of an intravascular blood pressure signal 122 and of a contact index 120 (derived from an electrode-tissue interface series resistance signal), as determined in accordance with an application of the present invention. These signals were measured during the same experiment as the data presented above in FIG. 3B. As can be seen, the contact index 120 (the quotient of (a) the electrode-tissue interface series resistance divided by (b) the electrode-tissue interface capacitance) varies cyclically, with a characteristic frequency corresponding to the cardiac cycle (approximately 1 Hz) and also with a smaller-magnitude characteristic frequency corresponding to the respiratory or ventilation cycle (approximately 0.2 Hz). The blood pressure signal 122 in FIG. 3C similarly shows the same two characteristic frequencies.

Reference is made to FIG. 3D, which shows a graph of the frequency domain 130 of intravascular blood pressure signal 122 and a graph of the frequency domain 132 of contact index 120, in accordance with an application of the present invention. (Signal 122 and index 120 are described hereinabove with reference to FIG. 3C.) For some applications, the correlation between intravascular blood pressure signal 122 and contact index 120 (or the electrode-tissue interface series resistance signal) is quantified by temporal cross-correlation (optionally, using the data as shown in FIG. 3C) or by matching main spectral harmonics of the signals. For example, control circuitry 70 may identify matching spectral harmonics representing respiration or ventilation (e.g., using a harmonic that is between 0.1 and 0.3 Hz) or matching spectral harmonics representing the cardiac cycle (e.g., using a harmonic that is between 0.7 and 2.0 Hz).

For some applications, control circuitry 70 is configured to (a) calculate the at least one time-varying component of the electrode-tissue impedance by calculating the electrode-tissue impedance, and (b) calculate the level of correlation between the electrode-tissue impedance and the periodic hemodynamic signal.

For some applications, the at least one time-varying component of the elect rode-tissue impedance includes the electrode-tissue interface series resistance. Control circuitry 70 is configured to (a) calculate the at least one time-varying component of the electrode-tissue impedance by calculating the electrode-tissue interface series resistance, and (b) calculate the level of correlation, between the electrode-tissue interface series resistance and the periodic hemodynamic signal.

For some applications, the at least one time-varying component of the electrode-tissue impedance includes the electrode-tissue interface series resistance and the electrode-tissue interface capacitance. Control circuitry 70 is configured to (a) calculate the at least one time-varying component of the electrode-tissue impedance by calculating the electrode-tissue impedance and the electrode-tissue interface capacitance, and a relationship between the electrode-tissue impedance and the electrode-tissue interface capacitance, and (b) calculate the level of correlation between (a) the relationship between the electrode-tissue impedance and the electrode-tissue interface capacitance and (b) the periodic hemodynamic signal.

For some applications, the at least one time-varying component of the electrode-tissue impedance includes the electrode-tissue interface capacitance. Control circuitry 70 is configured to (a) calculate the at least one time-varying component of the electrode-tissue impedance by calculating the electrode-tissue interface capacitance, and (b) calculate the level of correlation between the electrode-tissue interface capacitance and the periodic hemodynamic signal.

For some applications, control circuitry 70 is configured to activate the at least one intrarenal electrode 44 to apply an excitatory current to a renal nerve of the subject, in response ho the level of contact being at least the threshold level of contact. For example, control circuitry 70 may apply the excitatory current between (a) pair 42 of intrarenal electrodes 44, (b) the one of intrarenal electrodes 44 and intracorporeal reference electrode 50, or (c) the one of intrarenal electrodes 44 and external ground electrode 52. Control circuitry 70 may sense one or more physiological parameters of the subject in response to applications of the excitatory current, in order to further ascertain a level of contact between the at least one pair of electrodes and wall 110 of renal artery 108, and/or to ascertain a level of electrical coupling with the renal nerve, e.g., by sensing changes in blood pressure. For example, techniques may be used that are described in above-referenced International Application PCT/IB2015/053350.

For some applications, control circuitry 70 is configured to activate the at least one pair of electrodes (such as at least one pair 42 of intrarenal electrodes 44, at least one of intrarenal electrodes 44 and intracorporeal reference electrode 50, or at least one of intrarenal electrodes 44 and external, ground electrode 52) to ablate a renal nerve of the subject, in response to the level of contact being at least the threshold level of contact. For example, techniques may be used that are described in above-referenced International Application PCT/IB2015/053350.

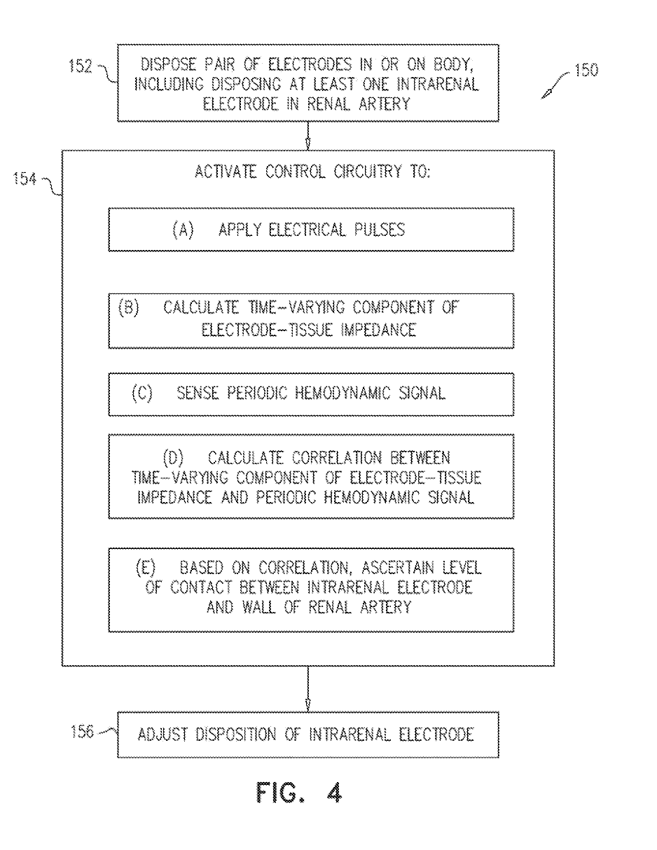

Reference is now made to FIG. 4, which is a flow chart that schematically illustrated a method 150 for ascertaining a level of contact between at least one intrarenal electrode 44 and wall 110 of renal artery 108, in accordance with an application of the present invention.

At an electrode disposition step 152, at least one pair of electrodes is disposed in or on the body of the subject, including disposing at least one intrarenal electrode 44 in renal artery 108. At an activation step 154, control circuitry 70 is activated to:

(a) apply electrical pulses between the pair of electrodes,

(b) calculate at least one time-varying component of electrode-tissue impedance based on applying the pulses,

(c) sense a periodic hemodynamic signal of the subject,

(d) calculate a level of correlation between the at least one time-varying component of the electrode-tissue impedance and the periodic hemodynamic signal, and

(e) based on the level of correlation, ascertain a level of contact between the at least one intrarenal electrode 44 and 110 wall of renal artery 108.

At a disposition adjustment step 186, in response to the level of contact being less than a threshold level of contact, a disposition of the at least one intrarenal electrode 44 in renal artery 108 is adjusted. The method may further comprise any of the techniques described hereinabove.

Reference is now made to FIG. 5, which is a schematic illustration of a system 180 for ablating and/or stimulating nerve tissue of a blood vessel of a subject, in accordance with an application of the present invention. Except as described hereinbelow, system 180 is generally similar to system 20 described hereinabove with reference to FIGS. 1-2B, and may incorporate any features thereof, mutatis mutandis. Intrarenal electrodes 18 are referred to as intrarenal current-application electrodes 44.

In addition the elements of system 20 that system 180 comprises, system 180 farther comprises two or more sensing electrodes 132, which are separate and distinct from intrarenal current-application electrodes 44. Sensing electrodes 182 are configured to be disposed in or on a body of the subject. Typically, the two or more sensing electrodes 182 comprise at least one external electrode 100, which is configured to be disposed on an external surface of shin of the subject, an, optionally one or more intracorporeal sensing electrodes (e.g., sensor 60). Alternatively, the two or more sensing electrodes 182 comprise two or more intracorporeal sensing electrodes (e.g., sensors 60), and, optionally, no external electrodes 190.

For some applications, control circuitry 70 is configured to:
apply an electrical current between the pair of current-application electrodes (which, as mentioned above, may comprise (a) pair 42 of intrarenal electrodes 44, (b) one of intrarenal electrodes 44 and intracorporeal reference electrode 50, or (c) one of intrarenal electrodes 44 and external ground electrode 52),
while applying the electrical current, sense an electrical signal between the two or more sensing electrodes 182, including the at least one external electrode 190, and
based on a property of the electrical signal, ascertain a level of contact between at least one intrarenal current-application electrode 44 and wall 110 of renal artery 108.

For some applications, user interface 72 is configured to output the level of contact.

For some applications, the at lease one external electrode 190 comprises at least two external electrodes 190, and control circuitry 70 is configured to sense the electrical signal between the at least two external electrodes 190. Typically, the at least two external electrodes 190 are disposed at least 1 cm apart. For some applications, external electrodes 190 are conventional electrocardiogram (ECG) electrodes, which may be positioned at one or more of the conventional ECG electrode locations on the body, and which may also be used to sense an ECG of the subject, and/or at one or more non-conventional ECG electrode locations, e.g., on the surface of the body near the artery in which the current-application electrodes are positioned, e.g., near the kidney, e.g., on the subject's back.

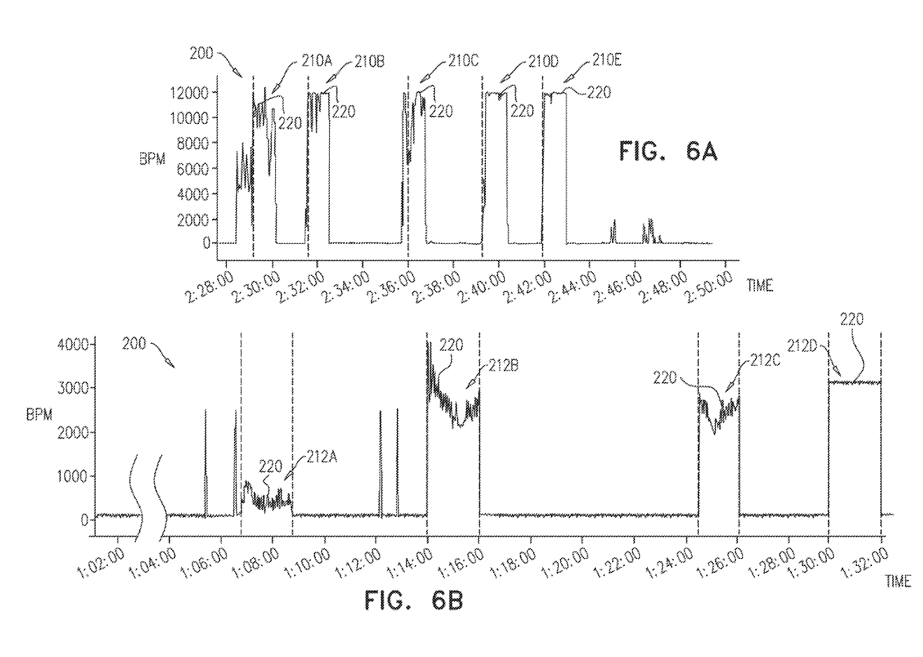

Reference is now made to FIGS. 6A-B, which are graphs 200 of signal rate values, sampled and calculated in accordance with an application of the present invention. FIGS. 6A-B show the signal rate (in beats per minutes) measured in an in vivo experiment conducted on behalf of the inventors in a female pig model, using a conventional ECG monitoring technique, which is one implementation of system 180, described hereinabove with reference to FIG. 5. FIG. 6A includes several periods 210 of application of nerve ablation, and FIG. 6B includes several periods 212 of electrical stimulation of the renal artery for purposes of sensing contact. The signal rate was automatically calculated by the conventional ECG monitor in the same manner as the ECG monitor conventionally calculates heart rate (typically by detecting R-waves and calculating the rate of the R-waves). In order to obtain the data presented in FIGS. 6A-B, a smoothing function was applied with the following properties: a sampling rate 1 kHz with an eleven-millisecond moving window. These graphs were recorded using an electrode device similar to intravascular device 1000 described in above-mentioned International Application PCT/IB2015/053350, and conventional ECG electrodes placed on the right front paw, left front paw, and abdomen, The nerve ablation signal was applied using a unipolar arrangement (between internal ablation electrodes and an external ground pad, at 460 kHz, at up to 8 W electric power per channel, with a sine waveform. The stimulation signal was applied with a rectangular waveform, at 20 Hz, a 2 ms pulse duration, up to 2 minutes stimulation, a 16 mA current amplitude, with a bipolar and biphasic signal.

Application of both the ablation and the stimulation signals “confuses” the ECG monitor, which senses the applied signals and interprets the applied signals as drastic increases in heart rates. Thus, the signal rate generated by the ECG monitor, and shown in graphs 200, reflects two unrelated phenomena:
during the periods in which the electrical current (whether an ablation current or a stimulation current) is not applied, the signal rate represents the subject's actual heart rate, as conventionally measured by the ECG monitor, and
during the periods in which the electrical current is applied, the signal rate represents the frequency of the applied electrical signal, as affected by the quality of contact with the at least one intrarenal electrode 44 and the wall of the renal artery, as measured by the ECG monitor; the signal rate thus reflects an artifact of the current application that is detected by the ECG monitor (for some applications, the signal rate is expressed in peaks per unit time).

It is this latter signal rate that is utilized in some applications of the present invention, as described below. In some applications of the present invention, the signal rate during the electrical-current application and non-current-application periods is calculated m the same manner (such as by an ECG monitoring technique). For example, control circuitry 70 may use (a) signals from the same electrodes to calculate the signal rate during both types of periods, and/or (b) the same features of the ECG signal for calculating the signal rates during both types of periods. For example, the rate of sensed “R-waves” may be used for calculating the signal rate, even though the sensed R-waves are not actually R-waves, but merely artifacts, during application of the electrical current.

The actual effectiveness of the ablation applied during each of periods 210 was assessed by performing histopathology post-ablation, and the actual effectiveness of the stimulation applied during each of periods 212 was assessed by observing the change in blood pressure caused by the stimulation at the locations of the electrodes compared to the blood pressure observed with the electrodes at approximately the same locations but with bad contact. In particular, (a) the ablation applied during periods 210A, 210B, and 210C was ineffective, and the ablation applied during periods 210D and 210E was effective, and (b) the stimulation applied during periods 212A, 212B, and 212C was ineffective, and the stimulation applied during period 212D was effective.

Effective application. of the current (whether ablation or stimulation) resulted in stable interference with the ECG. In some applications of the present invention, such stable interference during application of the electrical current is interpreted as an indication of good contact between the electrodes and tissue of the wall of the renal artery. (It is noted that such good tissue contact does not necessarily result in good nerve contact for ablation.)

For some applications, control circuitry 70 is configured to ascertain the level of contact based on a shape of graph 200 of the time-varying signal rate during application of the electrical current. For some applications, control circuitry 70 is configured to extract at least one plateau 220 from the graph 200, ascertain the shape of plateau 220, and ascertain the level of contact based on the shape of plateau 220. For some applications, control circuitry 70 is configured to calculate a flatness of plateau 220, and ascertain the level of contact based on the flatness of plateau 220. For example, the flatness of plateau 220 may be defined in terms of a least squares fit to a line. The flatness of plateau 220 may represent a stability of the time-varying signal rate during application of the current; for some applications, control circuitry 70 is configured to ascertain the level of contact based on the stability of the time-varying signal rate during application of the electrical current. For example, the stability may be calculated by comparing (a) an average variation and/or a maximum variation from an average value of the signal rate during application, of the electrical current with (b) a threshold value. For example, the threshold value may be 50 artifactual beats per minute.

For some applications, control circuitry 70 is configured to derive from the electrical signal a time-varying signal rate over time, and to ascertain the level of contact based on the time-varying signal rate. For some applications, control circuitry 70 applies a smoothing function to the signal rate (e.g., by averaging the signal rate over a moving window, e.g., having a duration of between 3 ms and 20 seconds, e.g., between 3-200 ms or 3-5000 ms, or sampling at between 3-200 points (e.g., 11 points) if, for example, the sampling rate is 1 kHz, e.g., separated by 3 to 200 ms, or a sampling rate of 5 to 333 Hz). For some applications, control circuitry 70 is configured to ascertain the level of contact based on a shape of graph 200 of the time-varying signal rate (e.g., a shape of the smoothed signal rate graph). For some applications, control circuitry 70 is configured to extract at least one plateau 220 from graph 200, ascertain the shape of plateau 220, and ascertain the level of contact based on the shape of plateau 220. For some applications, control circuitry 70 is configured to calculate a flatness of plateau 220, and ascertain the level of contact based on the flatness of plateau 220. The flatness of plateau 220 may, for example, be an indication of the strength of contact (i.e., the strength of mechanical pressure) and/or stability. The flatter plateau 220, the better the contact; conversely, the spikier plateau 220, the poorer the contact. For example, the flatness of plateau 220 may be defined in terms of a least squares fit to a line.

For some applications, the threshold level of flatness is preprogrammed in control circuitry 70. Alternatively, the threshold level of flatness is obtained during a procedure for each subject individually; for example, control circuitry 70 may set the threshold level of flatness daring deployment of electrode unit 30 in renal artery 106, or by temporarily deploying electrode unit 30 in the aorta and sensing the electrical signal while assuring that no contact is made between intrarenal electrodes and the wall of the aorta.

For some applications, control circuitry 70 is configured to derive from the electrical signal (e.g., the ECG signal) an actual heart rate of the subject during periods during which the electrical current is not applied, typically using the same electrodes used to derive the signal rate during application of the electrical current, and/or the same features of the ECG signal, e.g., the rate of “R-waves,” as described above. Control circuitry 70 may perform the same analysis to derive the heart rate as to derive the signal rate.

For some applications, control circuitry 70 is configured to ascertain the level of contact not responsively to an analysis of an amplitude of the sensed electrical signal. Unlike the other properties of the signal described herein, the amplitude of the electrical signal varies from subject to subject, and is thus generally not a good indicator of contact. Alternatively, for some applications, control circuitry 70 is configured to analyze the amplitude of the sensed electrical signal, for example by measuring an amplitude threshold in the same subject, and using both the amplitude and stability (flatness of the plateau) as criteria for contact quality. Higher amplitudes represent better contact between electrodes 44 and the wall of the renal artery, for a given subject, distance between the electrodes, and level of stimulation.

For some applications, control circuitry 70 is configured to sense a cardiac parameter (e.g., a parameter or an ECG) of the subject between the two or more sensing electrodes 182, including the at least one external electrode 190. For some applications, control circuitry 70 is configured to apply the electrical current as an excitatory electrical current to a renal nerve of the subject, and to ascertain the level of contact between pair 42 of intrarenal current-application electrodes 44 based additionally on the sensed cardiac parameter. For some applications, control circuitry 70 is configured to (a) apply she electrical current as an excitatory electrical current to a renal nerve of the subject, and (b) ascertain a level of suitability of a location of pair 42 of intrarenal current-application electrodes 44 along renal artery 108 based On the sensed cardiac parameter, and user interface 72 is configured to output information based on the level of suitability.

For some applications, control circuitry 70 is configured to (a) apply the electrical current as an excitatory electrical current to a renal, nerve of the subject, and (b) ascertain whether the subject is a suitable candidate for renal nerve ablation based on the sensed cardiac parameter, and user interface 72 is configured to (a) output an indication regarding whether the subject is a suitable candidate, and/or (b) activate the at least one intrarenal current-application electrode 44 to ablate a renal nerve of the subject, in response to ascertaining that the subject is a suitable candidate.

For some applications, control circuitry 70 is configured to apply the electrical current as an excitatory electrical current, such as described hereinabove with reference to FIGS. 2A-B. For some applications, control circuitry 70 is configured to apply the electrical current with a strength sufficient to ablate a renal nerve of the subject, such as described hereinabove with reference to FIGS. 2A-B.

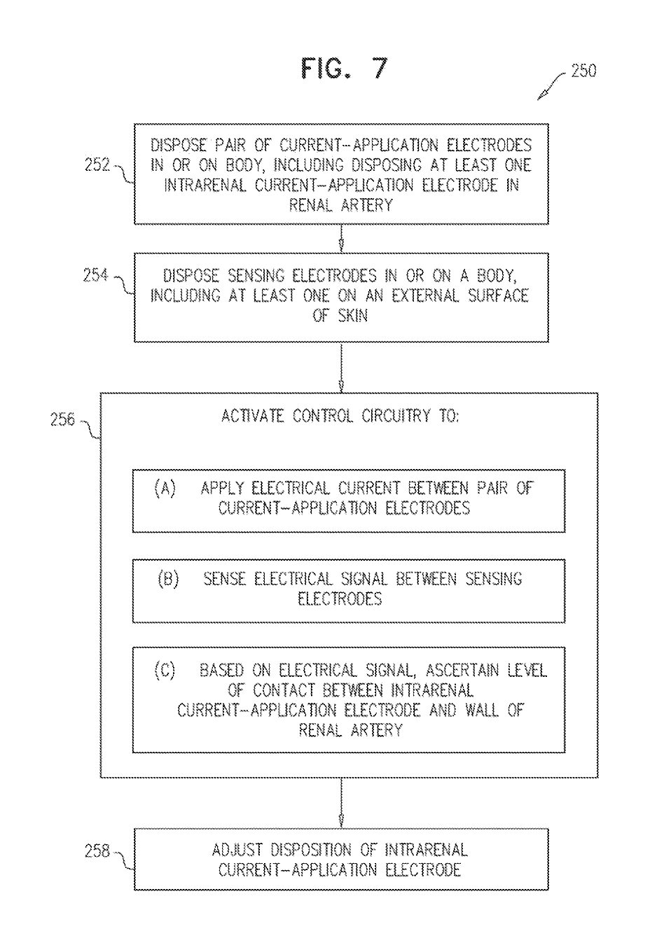

Reference is now made to FIG. 7, which is a flow chart that schematically illustrated a method 250 for ascertaining a level of contact between at least one intrarenal electrode and wall 110 of renal artery 108, in accordance with an application of the present invention.

At a current-application electrode disposition step 252, at least one pair of current-application electrodes 44 in or on a body of the subject, including disposing at least one intrarenal cur rent-application electrode 44 of the pair in renal artery 108. At a sensing electrode disposition step 254, two or more sensing electrodes 102 are disposed, in or on a body of the subject, typically including disposing at least one of sensing electrodes 182 on an external surface of skin of the subject, sensing electrodes 182 separate and distinct from the at least one intrarenal cur rent-application electrode 44. Alternatively, all of sensing electrodes 182 are disposed in the body of the subject, such as on electrode unit 30 (e.g., on central shaft 48) and/or on elongate shaft 40. Further alternatively or additionally, one or more of intrarenal current-application electrodes 44 also serve as one or more of sensing electrodes 182.

At an activation step 256, control circuitry 70 is activated to:

(a) apply an electrical current between the pair of current-application electrodes,

(b) while applying the electrical current, sense an electrical signal between the two or more sensing electrodes 182, including the as least one of the sensing electrodes 182 disposed on the external surface of the skin, and

(c) based on the electrical signal, ascertain a level of contact between the at least one intrarenal current-application electrode 44 and well 110 of renal artery 108.

At a disposition adjustment step 158, in response to the level of contact being less than a threshold level of contact, a disposition of the at least intrarenal current-application electrode in renal artery 108 is

adjusted. The method may further comprise any of the techniques described hereinabove.

Reference is again made to FIGS. 1-2B. In some applications of the present invention, control circuitry 70 is configured to, while applying an electrical current using at least one of intrarenal electrodes 44, derive an ECG signal, optionally using one or more of intrarenal electrodes 44 (e.g., two or more, or all), and, optionally, one or more other electrodes described herein, such as intracorporeal reference electrode 50, external ground electrode 52, and/or external electrode 190. Control circuitry 70 is configured to evaluate a level of quality of the ECG signal, for example, by evaluating an amplitude, shape, and/or both amplitude and shape of one or more features of she ECG signal, for example as described hereinabove with reference to FIGS. 5-7. Typically, the one or more intrarenal electrodes 44 used to sense the ECG are different from the at least one intrarenal electrode 44 used for applying the electrical current. Based on the level of quality of the ECG signal, control circuitry 70 ascertains a level of contact between the one or sore intrarenal electrodes 44 and wall 110 or renal artery 108. For example, control circuitry 70 may be configured to derive, from the ECG signal, a time-varying signal rate of the subject over a period of application of the electrical signal, and to ascertain the level of contact based on a stability of the time-varying signal rate. In response to the level of contact being at least a threshold level of contact, control circuitry 70 is configured to activate the one or more intrarenal electrodes 44 to apply an excitatory current to the renal nerve, and/or to ablate the renal nerve, as described hereinabove.

Although the techniques described herein have been generally described as being used in the renal artery, they may also be used in other arteries, blood vessels, or body lumen of the body, mutatis mutandis.

The scope of the present invention includes embodiments described in the following applications, which are assigned to the assignee of the present application and are incorporated herein by reference. In an embodiment, techniques and apparatus described in one or more of the following applications are combined with techniques and apparatus described herein:
US Provisional Application 61/722,293, filed Nov. 5, 2012
U.S. application Ser. No. 13/771,853, filed Feb. 20, 2013
US Provisional Application 61/811,880, filed Apr. 15, 2013

US Provisional Application 61/841,485, filed Jul. 1, 2013

US Provisional Application 61/862,561, filed Aug. 6, 2013

International Application PCT/IL2013/050903, filed Nov. 3 2013, which published as WO 2014/068577, and U.S. application Ser. No. 14/440,431, filed May 4, 2015, in the national stage thereof

US Provisional Application 61/989,741, riled May 7, 2014

International Application PCT/IB2015/053350, filed May 7, 2015

US Provisional Application 62/158,139, filed May 7, 2015

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1-21. (canceled)

22. A method comprising:
disposing at least one pair of current-application electrodes in or on a body of a subject, including disposing at least one intrarenal current-application electrode of the pair in a renal artery of the subject;
disposing two or more sensing electrodes in or on the body of the subject, including disposing at least one of the sensing electrodes on an external surface of skin of the subject, the sensing electrodes separate and distinct from the at least one intrarenal current-application electrode;
activating control circuitry to:
(a) apply an electrical current between the pair of current-application electrodes,
(b) while applying the electrical current, sense an electrical signal between the two or more sensing electrodes, including the at least one of the sensing electrodes disposed on the external surface of the skin, and
(c) based on the electrical signal, ascertain a level of contact between the at least one intrarenal current-application electrode and a wall of the renal artery; and
in response to the level of contact being less than a threshold level of contact, adjusting a disposition of the at least one intrarenal current-application electrode in the renal artery.

23. The method according to claim 22, wherein disposing the two or more sensing electrodes in or on the body of the subject comprises disposing at least two of the sensing electrodes on the external surface of the skin, and wherein activating the control circuitry comprises activating the control circuitry to sense the electrical signal between the at least two of the sensing electrodes disposed on the external surface of the skin.

24-26. (canceled)

27. The method according to claim 22, wherein activating the control circuitry comprises activating the control circuitry to ascertain the level of contact based on a shape of the electrical signal.

28. The method according to claim 27, wherein activating the control circuitry comprises activating the control circuitry to:
extract at least one plateau from the electrical signal,
ascertain the shape of the plateau, and
ascertain the level of contact based on the shape of the plateau.

29. The method according to claim 28, wherein activating the control circuitry comprises activating the control circuitry to:
calculate a flatness of the plateau, and
ascertain the level of contact based on the flatness of the plateau.

30. The method according to claim 22, wherein activating the control circuitry comprises activating the control circuitry to derive from the electrical signal a time-varying signal rate over time, and to ascertain the level of contact based on the time-varying signal rate.

31. The method according to claim 30, wherein activating the control circuitry comprises activating the control circuitry to, while not applying the electrical current:
sense the electrical signal between the two or more sensing electrodes, including the at least one of the sensing electrodes disposed on the external surface of the skin, and
ascertain a heart rate of the subject from the electrical signal.

32. The method according to claim 30, wherein activating the control circuitry comprises activating the control circuitry to ascertain the level of contact based on a stability of the time-varying signal rate.

33-35. (canceled)

36. The method according to claim 22, wherein the method further comprises activating the control circuitry to sense a cardiac parameter of the subject between the two or more sensing electrodes, including the at least one of the sensing electrodes disposed on the external surface of the skin.

37-39. (canceled)

40. The method according to claim 22, wherein activating the control circuitry to apply the electrical current comprises activating the control circuitry to apply an excitatory electrical current to a renal nerve of the subject.

41. The method according to claim 22, wherein activating the control circuitry to apply the electrical current comprises activating the control circuitry to apply the electrical current with a strength sufficient to ablate a renal nerve of the subject.

42. A method comprising:
disposing a plurality of electrodes in or on a body of a subject, including disposing two or more intrarenal electrodes of the plurality of electrodes in a renal artery of the subject;
activating control circuitry to:
(a) apply an electrical current between a pair of the electrodes, including at least a first one of the intrarenal electrodes,
(b) while applying the electrical current, sense an electrocardiogram (ECG) signal using the plurality of electrodes, including at least a second one of the intrarenal electrodes,
(c) evaluate a level of quality of the ECG signal, and
(d) based on the level of quality, ascertain a level of contact between the at least one intrarenal electrode and a wall of the renal artery; and
in response to the level of contact being less than a threshold level of contact, adjusting a disposition of the at least one intrarenal electrode in the renal artery.

43. The method according to claim 42,
wherein disposing the plurality of electrodes comprises disposing at least one external electrode of the plurality of electrodes on an external surface of skin of the subject, and
wherein activating the control circuitry comprises activating the control circuitry to sense the ECG signal using the plurality of electrodes, including the at least one intrarenal electrode and the at least one external electrode.

44-46. (canceled)

47. The method according to claim 42, wherein activating the control circuitry comprises activating the control circuitry to derive from the ECG signal a time-varying signal rate over time, and to ascertain the level of contact based on the time-varying signal rate.

48. The method according to claim 47, wherein activating the control circuitry comprises activating the control circuitry to, while not applying the electrical current:
sense the ECG signal using the plurality of electrodes, including the at least a second one of the intrarenal electrodes, and
ascertain a heart rate of the subject from the electrical signal.

49. The method according to claim 47, wherein activating the control circuitry comprises activating the control circuitry to ascertain the level of contact based on a stability of the time-varying signal rate.

50. The method according to claim 47, wherein activating the control circuitry comprises activating the control circuitry to ascertain the level of contact based on a shape of the time-varying signal rate.

51-57. (canceled)

58. A method comprising:
disposing a plurality of electrodes in or on a body of a subject, including disposing at least one intrarenal electrode of the plurality of electrodes in a renal artery of the subject;
activating control circuitry to:
(a) apply an electrical current between a pair of the electrodes, including at least one of the intrarenal electrodes,
(b) while applying the electrical current, sense an electrocardiogram (ECG) signal using the plurality of electrodes,
(c) derive a time-varying signal rate from the ECG signal, and
(d) based on a stability of the time-varying signal rate, ascertain a level of contact between the at least one intrarenal electrode and a wall of the renal artery; and
in response to the level of contact being less than a threshold level of contact, adjusting a disposition of the at least one intrarenal electrode in the renal artery.

59-84. (canceled)

85. Apparatus comprising:
at least one pair of current-application electrodes, which comprise at least one intrarenal electrode configured to be disposed in a renal artery of a subject;
two or more sensing electrodes, which are separate and distinct from the intrarenal current-application electrodes, and which are configured to be disposed in or on a body of the subject, wherein the two or more sensing electrodes comprise at least one external electrode, which is configured to be disposed on an external surface of skin of the subject;
control circuitry, configured to:
(a) apply an electrical current between the pair of current-application electrodes,
(b) while applying the electrical current, sense an electrical signal between the two or more sensing electrodes, including the at least one external electrode, and
(c) based on a property of the electrical signal, ascertain a level of contact between the at least one intrarenal current-application electrode and a wall of the renal artery; and
a user interface, which is configured to output the level of contact.

86. The apparatus according to claim 85, wherein the at least one external electrode comprises at least two external electrodes, and wherein the control circuitry is configured to sense the electrical signal between the at least two external electrodes.

87-89. (canceled)

90. The apparatus according to claim 85, wherein the control circuitry is configured to ascertain the level of contact based on a shape of the electrical signal.

91. The apparatus according to claim 90, wherein the control circuitry is configured to:
extract at least one plateau from the electrical signal,
ascertain the shape of the plateau, and
ascertain the level of contact based on the shape of the plateau.

92. The apparatus according to claim 91, wherein the control circuitry is configured to:
calculate a flatness of the plateau, and
ascertain the level of contact based on the flatness of the plateau.

93. The apparatus according to claim 85, wherein the control circuitry is configured to derive from the electrical signal a time-varying signal rate over time, and to ascertain the level of contact based on the time-varying signal rate.

94. The apparatus according to claim 93, wherein the control circuitry is configured to, while not applying the electrical current:
sense the electrical signal between the two or more sensing electrodes, including the two or more sensing electrodes, including the at least one external electrode, and
ascertain a heart rate of the subject from the electrical signal.

95. The apparatus according to claim 93, wherein the control circuitry is configured to ascertain the level of contact based on a stability of the time-varying signal rate.

96-98. (canceled)

99. The apparatus according to claim 85, wherein the control circuitry is configured to sense a cardiac parameter of the subject between the two or more sensing electrodes, including the at least one external electrode.

100-103. (canceled)

104. The apparatus according to claim 85, wherein the control circuitry is configured to apply the electrical current as an excitatory electrical current.

105. The apparatus according to claim 85, wherein the control circuitry is configured to apply the electrical current with a strength sufficient to ablate a renal nerve of the subject.

106-127. (canceled)
